(19) United States
(12) Patent Application Publication
(10) Pub. No.: US 2012/0253140 A1
(43) Pub. Date: Oct. 4, 2012

(54) Systems And Methods For Autonomic Nervous System Monitoring

(75) Inventors: Paul S. Addison, Edinburgh (GB); James N. Watson, Dunfermline (GB)

(73) Assignee: Nellcor Puritan Bennett Ireland, Galway (IE)

(21) Appl. No.: 13/076,281

(22) Filed: Mar. 30, 2011

(51) Int. Cl. A61B 5/00; A61B 5/08

(52) U.S. Cl. 600/301; 600/529

(57) ABSTRACT

Methods and systems are disclosed for determining physiological information about a patient's autonomic nervous system based on at least one physiological signal measured from the patient and at least one known characteristic of a patient's respiration. Respiration protocol may be provided to guide characteristics of the patient's respiration. The physiological signal measured from the patient may be transformed using a wavelet transform to create a transformed signal, and a scalogram may be generated based at least in part on the transformed signal. A metric that may indicate information about the patient's autonomic nervous system may be determined from the scalogram and the known characteristic of the patient's respiration.

### SUMMARY

The present disclosure relates to physiological signal processing, and more particularly, the present disclosure relates to physiological signal processing to determine information about a patient's autonomic nervous system (ANS).

Respiration may modulate aspects of a patient's physiology, such as heart rate. This modulation of the heart rate is known as respiratory sinus arrhythmia (RSA), which may be an indicator of autonomic neurological physical mechanisms. For example, weakened RSA may occur in patients with malignant arrhythmias who survive cardiopulmonary resuscitation and patients with coronary artery disease (who may have no history of acute myocardial infarction or congestive heart failure). In diabetic patients, diminished RSA may be a sensitive marker to autonomic neuropathy. RSA is typically prominent in infants, athletes, and adults who regularly exercise. There is, therefore, a need for the capability to monitor RSA as a marker of autonomic function.

Many causes of naturally occurring heart rate variability (HRV), including RSA, may occur simultaneously and result in a signal that represents a complex mix of these influences, which may represent a challenge for HRV analysis. For example, one main factor that may influence HRV, respiration, may vary from patient to patient in terms of, for example, respiration rate and the variation in respiration rate for each patient. HRV may also be affected by, for example, tidal volume and lung compliance, the patient changing posture, undertaking physical activity, administration of medication, or any combination thereof.

In some embodiments, methods and systems are provided for the analysis of ANS activity through the controlled testing and parameterization of RSA. RSA may be quantified for a patient undergoing paced respiration (i.e., one or more aspects of a patient's respiratory activity is directed according to, for example, clinical instruction, or otherwise controlled). By pacing respiration over a number of breaths, it may be possible to characterize RSA in terms of one or more metrics that may be monitored for changes resulting from the controlled respiratory activity. Some illustrative metrics include, for example, the phase relationship between some fiducial point or points in the respiratory cycle and the heart rate (or changes thereof) (e.g., the response time for the heart rate to increase after the onset of inspiration), a maximum heart rate, a minimum heart rate, a mean heart rate, any other suitable metric or measurable signal morphology, or any combination thereof.

In an embodiment the respiration rate may be slowly varied over time (e.g., by instructing the patient accordingly) and a corresponding change in ANS parameters may be measured. In some embodiments, the response to sudden respiration rate changes, apnea (i.e., when detected), single large breaths, or a combination thereof may be tested for effects on ANS by measuring any suitable metrics or measurable signal morphologies.

For example, to monitor respiration and heart rate variation, one or more suitable physiological signals, such as a photoplethysmograph (PPG) signal, may be analyzed. Any suitable software, hardware, or both may use one or more signal processing steps to determine useful information from a physiological signal.

In an embodiment, the PPG signal may be transformed using, for example, a wavelet transform, a frequency domain transform (e.g., Fourier transform), any other suitable transform, or any combination thereof. In the context of a wavelet transform, for example, a scalogram generated from the transformed PPG signal may exhibit a first band of interest and a second band of interest that may respectively represent pulse components and breathing components. Morphologies of the pulse band, including, for example, RSA may be monitored in response to controlled respiratory activity of the patient in order to gather information about, for example, the patient ANS.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIGS. 3(a) and 3(b) show illustrative views of a scalogram derived from a PPG signal in accordance with an embodiment;

FIG. 3(c) shows an illustrative scalogram derived from a signal containing two pertinent components in accordance with an embodiment;

FIG. 3(d) shows an illustrative schematic of signals associated with a ridge in FIG. 3(c) and illustrative schematics of a further wavelet decomposition of these associated signals in accordance with an embodiment;

FIGS. 3(e) and 3(f) are flow charts of illustrative steps involved in performing an inverse continuous wavelet transform in accordance with embodiments;

FIG. 4 is a block diagram of an illustrative continuous wavelet processing system in accordance with some embodiments;

FIG. 5 is an illustrative signal which may be analyzed in accordance with an embodiment;

FIG. 6 are illustrative plots showing a relationship between respiratory activity and heart rate in accordance with an embodiment;

FIG. 7 is an illustrative scalogram showing a relationship between respiratory activity and heart rate in accordance with an embodiment;

FIG. 8 is an enlarged view of an illustrative scalogram showing a relationship between respiratory activity and heart rate in accordance with an embodiment; and

FIG. 9 is a flow chart of an illustrative process for determining information about a patient's autonomic nervous system in accordance with an embodiment.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.

1. First, the natural logarithm of (1) is taken (“log” will be used to represent the natural logarithm) for IR and Red

log I=log Iₒ−(sβₒ+(1−s)βᵣ)l (2)

2. (2) is then differentiated with respect to time

$$\frac{d log I}{d t} = - ( s {β}_{o} + ( 1 - s ) {β }_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{O} ( {λ}_{R} ) + ( 1 - s ) {β}_{r} ( {λ}_{R} )}{s {β}_{O} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\frac{d log I ( {λ}_{R} )}{d t} ( {β}_{O} ( {λ}_{IR} ) - {β}_{r} ( {λ}_{IR} ) ) - \frac{d log I ( {λ}_{IR} )}{d t} ( {β}_{O} ( {λ}_{R} ) - {β}_{r} ( {λ}_{R} ) )}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{O} ( {λ}_{IR} ) - {β}_{r} ( {λ}_{IR} ) ) - {β}_{O} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t) (8)

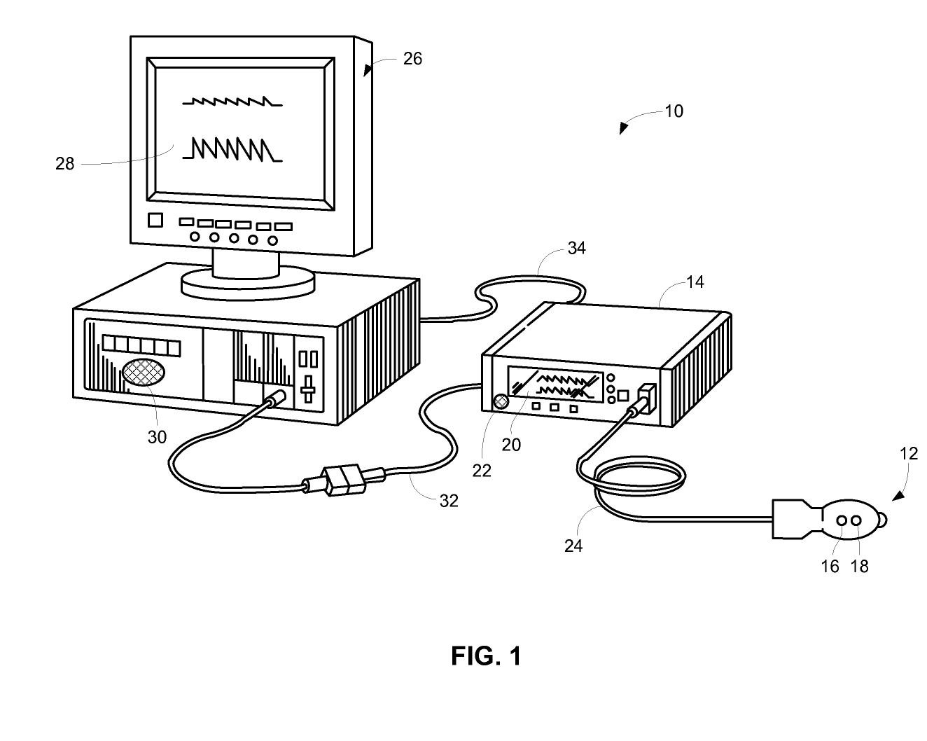

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

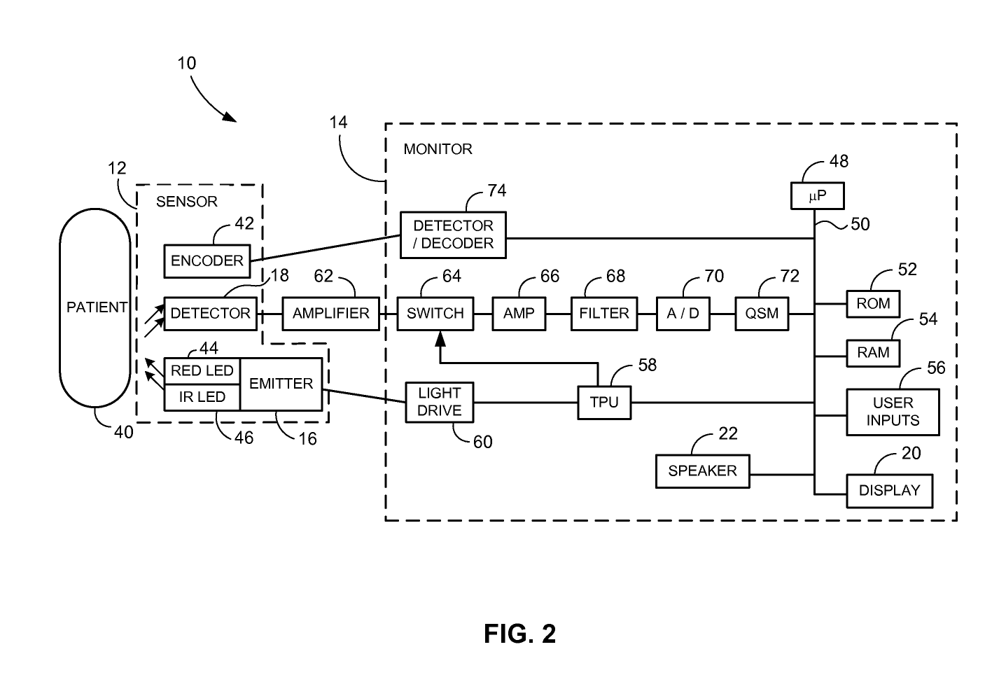

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. In an embodiment, microprocessor 48 may be used for signal processing. For example, microprocessor 48 may tune a wavelet transform to a particular characteristic frequency in a scale band of interest. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In one embodiment, a PPG signal may be transformed using a continuous wavelet transform. Information derived from the transform of the PPG signal (i.e., in wavelet space) may be used to provide measurements of one or more physiological parameters.

The continuous wavelet transform of a signal x(t) in accordance with the present disclosure may be defined as

$$T ( a , b ) = \frac{1}{\sqrt{a}} {\int }_{- ∞}^{+ ∞} x ( t ) {ψ}^{*} ( \frac{t - b}{a} ) d t \qquad ( 9 )$$

where ψ*(t) is the complex conjugate of the wavelet function ψ(t), a is the dilation parameter of the wavelet and b is the location parameter of the wavelet. The transform given by equation (9) may be used to construct a representation of a signal on a transform surface. The transform may be regarded as a time-scale representation. Wavelets are composed of a range of frequencies, one of which may be denoted as the characteristic frequency of the wavelet, where the characteristic frequency associated with the wavelet is inversely proportional to the scale a. One example of a characteristic frequency is the dominant frequency. Each scale of a particular wavelet may have a different characteristic frequency. The underlying mathematical detail required for the implementation within a time-scale can be found, for example, in Paul S. Addison, The Illustrated Wavelet Transform Handbook (Taylor & Francis Group 2002), which is hereby incorporated by reference herein in its entirety.

The continuous wavelet transform decomposes a signal using wavelets, which are generally highly localized in time. The continuous wavelet transform may provide a higher resolution relative to discrete transforms, thus providing the ability to garner more information from signals than typical frequency transforms such as Fourier transforms (or any other spectral techniques) or discrete wavelet transforms. Continuous wavelet transforms allow for the use of a range of wavelets with scales spanning the scales of interest of a signal such that small scale signal components correlate well with the smaller scale wavelets and thus manifest at high energies at smaller scales in the transform. Likewise, large scale signal components correlate well with the larger scale wavelets and thus manifest at high energies at larger scales in the transform. Thus, components at different scales may be separated and extracted in the wavelet transform domain. Moreover, the use of a continuous range of wavelets in scale and time position allows for a higher resolution transform than is possible relative to discrete techniques.

In addition, transforms and operations that convert a signal or any other type of data into a spectral (i.e., frequency) domain necessarily create a series of frequency transform values in a two-dimensional coordinate system where the two dimensions may be frequency and, for example, amplitude. For example, any type of Fourier transform would generate such a two-dimensional spectrum. In contrast, wavelet transforms, such as continuous wavelet transforms, are required to be defined in a three-dimensional coordinate system and generate a surface with dimensions of time, scale and, for example, amplitude. Hence, operations performed in a spectral domain cannot be performed in the wavelet domain; instead the wavelet surface must be transformed into a spectrum (i.e., by performing an inverse wavelet transform to convert the wavelet surface into the time domain and then performing a spectral transform from the time domain). Conversely, operations performed in the wavelet domain cannot be performed in the spectral domain; instead a spectrum must first be transformed into a wavelet surface (i.e., by performing an inverse spectral transform to convert the spectral domain into the time domain and then performing a wavelet transform from the time domain). Nor does a cross-section of the three-dimensional wavelet surface along, for example, a particular point in time equate to a frequency spectrum upon which spectral-based techniques may be used. At least because wavelet space includes a time dimension, spectral techniques and wavelet techniques are not interchangeable. It will be understood that converting a system that relies on spectral domain processing to one that relies on wavelet space processing would require significant and fundamental modifications to the system in order to accommodate the wavelet space processing (e.g., to derive a representative energy value for a signal or part of a signal requires integrating twice, across time and scale, in the wavelet domain while, conversely, one integration across frequency is required to derive a representative energy value from a spectral domain). As a further example, to reconstruct a temporal signal requires integrating twice, across time and scale, in the wavelet domain while, conversely, one integration across frequency is required to derive a temporal signal from a spectral domain. It is well known in the art that, in addition to or as an alternative to amplitude, parameters such as energy density, modulus, phase, among others may all be generated using such transforms and that these parameters have distinctly different contexts and meanings when defined in a two-dimensional frequency coordinate system rather than a three-dimensional wavelet coordinate system. For example, the phase of a Fourier system is calculated with respect to a single origin for all frequencies while the phase for a wavelet system is unfolded into two dimensions with respect to a wavelet's location (often in time) and scale.

The energy density function of the wavelet transform, the scalogram, is defined as

S(a,b)=|T(a,b)|² (10)

where ‘∥’ is the modulus operator. The scalogram may be resealed for useful purposes. One common resealing is defined as

$${S}_{R} ( a , b ) = \frac{{\vert T ( a , b ) \vert}^{2}}{a} \qquad ( 11 )$$

and is useful for defining ridges in wavelet space when, for example, the Morlet wavelet is used. Ridges are defined as the locus of points of local maxima in the plane. Any reasonable definition of a ridge may be employed in the method. Also included as a definition of a ridge herein are paths displaced from the locus of the local maxima. A ridge associated with only the locus of points of local maxima in the plane are labeled a “maxima ridge”.

For implementations requiring fast numerical computation, the wavelet transform may be expressed as an approximation using Fourier transforms. Pursuant to the convolution theorem, because the wavelet transform is the cross-correlation of the signal with the wavelet function, the wavelet transform may be approximated in terms of an inverse FFT of the product of the Fourier transform of the signal and the Fourier transform of the wavelet for each required a scale and then multiplying the result by \sqrt{a}.

In the discussion of the technology which follows herein, the “scalogram” may be taken to include all suitable forms of resealing including, but not limited to, the original unsealed wavelet representation, linear resealing, any power of the modulus of the wavelet transform, or any other suitable resealing. In addition, for purposes of clarity and conciseness, the term “scalogram” shall be taken to mean the wavelet transform, T(a,b) itself, or any part thereof. For example, the real part of the wavelet transform, the imaginary part of the wavelet transform, the phase of the wavelet transform, any other suitable part of the wavelet transform, or any combination thereof is intended to be conveyed by the term “scalogram”.

A scale, which may be interpreted as a representative temporal period, may be converted to a characteristic frequency of the wavelet function. The characteristic frequency associated with a wavelet of arbitrary a scale is given by

$$f = \frac{{f}_{c}}{a} \qquad ( 12 )$$

where f_{c}, the characteristic frequency of the mother wavelet (i.e., at a=1), becomes a scaling constant and f is the representative or characteristic frequency for the wavelet at arbitrary scale a.

Any suitable wavelet function may be used in connection with the present disclosure. One of the most commonly used complex wavelets, the Morlet wavelet, is defined as:

ψ(t)=π^{-1/4}(e^{i2πf}^{₀}^{t}−e^{-(2πf}^{₀}⁾^{²}^{/2})e^{-t}^{²}^{/2} (13)

where f₀ is the central frequency of the mother wavelet. The second term in the parenthesis is known as the correction term, as it corrects for the non-zero mean of the complex sinusoid within the Gaussian window. In practice, it becomes negligible for values of f₀>>0 and can be ignored, in which case, the Morlet wavelet can be written in a simpler form as

$$ψ ( t ) = \frac{1}{{π}^{1 / 4}} {e}^{i 2 π {f}_{0} t} {e}^{- {t}^{2} / 2} \qquad ( 14 )$$

This wavelet is a complex wave within a scaled Gaussian envelope. While both definitions of the Morlet wavelet are included herein, the function of equation (14) is not strictly a wavelet as it has a non-zero mean (i.e., the zero frequency term of its corresponding energy spectrum is non-zero). However, it will be recognized by those skilled in the art that equation (14) may be used in practice with f₀>>0 with minimal error and is included (as well as other similar near wavelet functions) in the definition of a wavelet herein. A more detailed overview of the underlying wavelet theory, including the definition of a wavelet function, can be found in the general literature. Discussed herein is how wavelet transform features may be extracted from the wavelet decomposition of signals. For example, wavelet decomposition of PPG signals may be used to provide clinically useful information within a medical device.

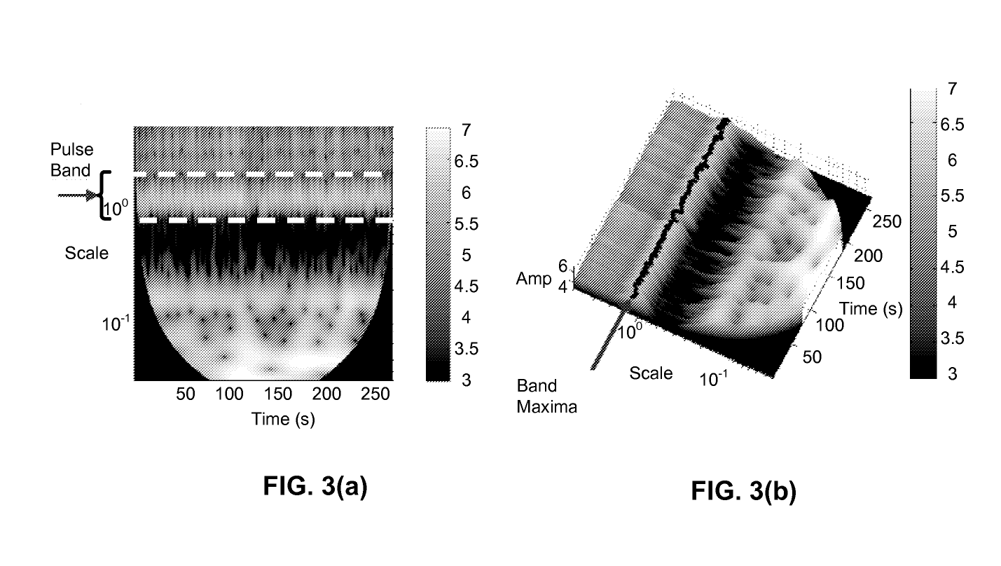

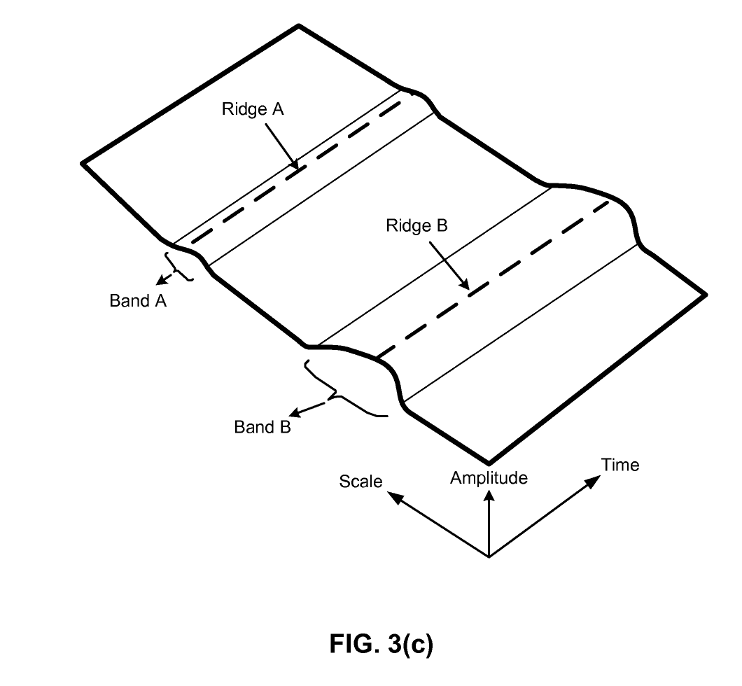

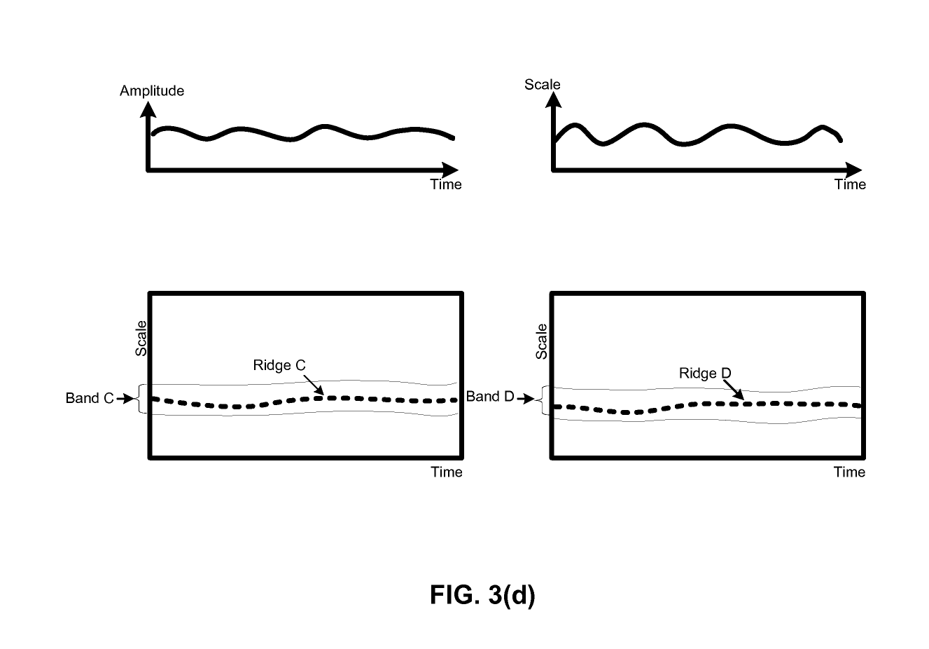

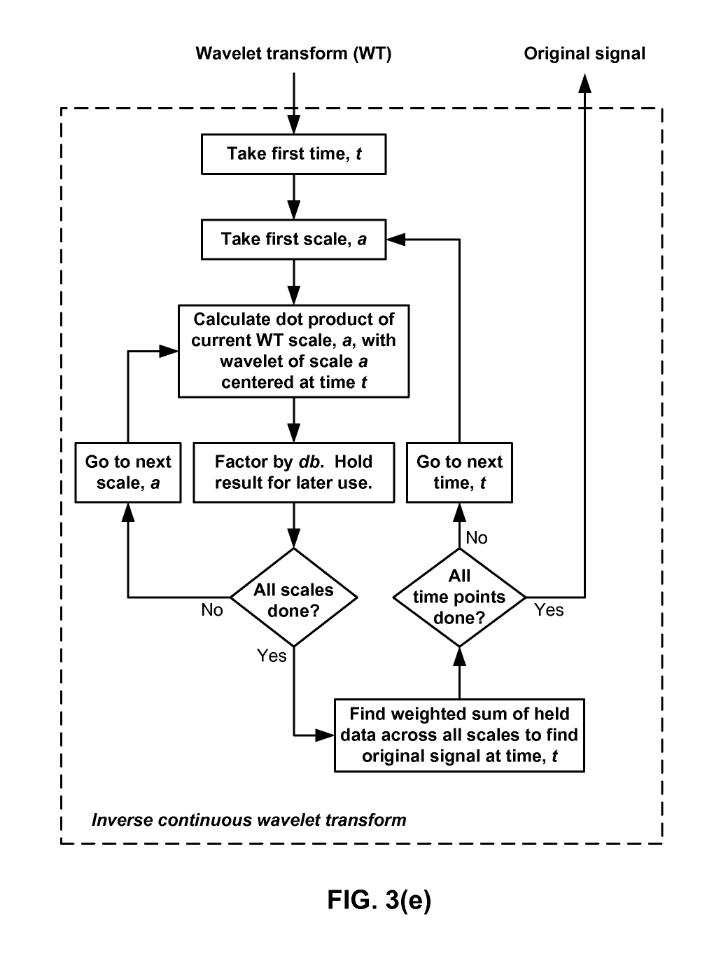

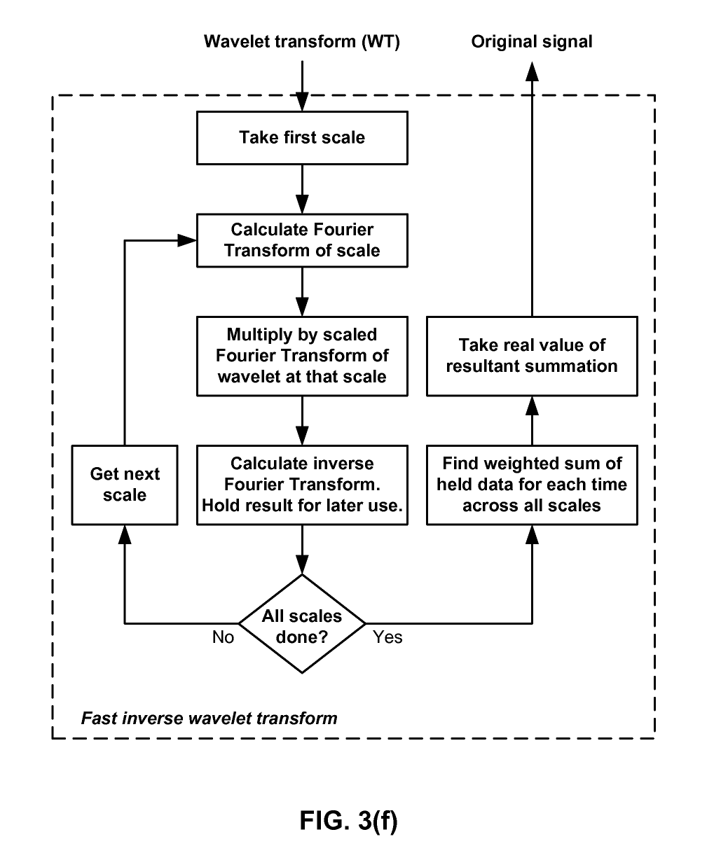

Pertinent repeating features in a signal give rise to a time-scale band in wavelet space or a resealed wavelet space. For example, the pulse component of a PPG signal produces a dominant band in wavelet space at or around the pulse frequency. FIGS. 3(a) and (b) show two views of an illustrative scalogram derived from a PPG signal, according to an embodiment. The figures show an example of the band caused by the pulse component in such a signal. The pulse band is located between the dashed lines in the plot of FIG. 3(a). The band is formed from a series of dominant coalescing features across the scalogram. This can be clearly seen as a raised band across the transform surface in FIG. 3(b) located within the region of scales indicated by the arrow in the plot (corresponding to 60 beats per minute). The maxima of this band with respect to scale is the ridge. The locus of the ridge is shown as a black curve on top of the band in FIG. 3(b). By employing a suitable resealing of the scalogram, such as that given in equation (11), the ridges found in wavelet space may be related to the instantaneous frequency of the signal. In this way, the pulse rate may be obtained from the PPG signal. Instead of resealing the scalogram, a suitable predefined relationship between the scale obtained from the ridge on the wavelet surface and the actual pulse rate may also be used to determine the pulse rate.

By mapping the time-scale coordinates of the pulse ridge onto the wavelet phase information gained through the wavelet transform, individual pulses may be captured. In this way, both times between individual pulses and the timing of components within each pulse may be monitored and used to detect heart beat anomalies, measure arterial system compliance, or perform any other suitable calculations or diagnostics. Alternative definitions of a ridge may be employed. Alternative relationships between the ridge and the pulse frequency of occurrence may be employed. As discussed below, the wavelet transform may be tuned to a separate characteristic frequency in the location of each ridge.

As discussed above, pertinent repeating features in the signal give rise to a time-scale band in wavelet space or a resealed wavelet space. For a periodic signal, this band remains at a constant scale in the time-scale plane. For many real signals, especially biological signals, the band may be non-stationary; varying in scale, amplitude, or both over time. FIG. 3(c) shows an illustrative schematic of a wavelet transform of a signal containing two pertinent components leading to two bands in the transform space, according to an embodiment. These bands are labeled band A and band B on the three-dimensional schematic of the wavelet surface. In this embodiment, the band ridge is defined as the locus of the peak values of these bands with respect to scale. For purposes of discussion, it may be assumed that band B contains the signal information of interest. This will be referred to as the “primary band”. In addition, it may be assumed that the system from which the signal originates, and from which the transform is subsequently derived, exhibits some form of coupling between the signal components in band A and band B. When noise or other erroneous features are present in the signal with similar spectral characteristics of the features of band B then the information within band B can become ambiguous (i.e., obscured, fragmented or missing). In this case, the ridge of band A may be followed in wavelet space and extracted either as an amplitude signal or a scale signal which will be referred to as the “ridge amplitude perturbation” (RAP) signal and the “ridge scale perturbation” (RSP) signal, respectively. The RAP and RSP signals may be extracted by projecting the ridge onto the time-amplitude or time-scale planes, respectively. The top plots of FIG. 3(d) show a schematic of the RAP and RSP signals associated with ridge A in FIG. 3(c). Below these RAP and RSP signals are schematics of a further wavelet decomposition of these newly derived signals. This secondary wavelet decomposition allows for information in the region of band B in FIG. 3(c) to be made available as band C and band D. The ridges of bands C and D may serve as instantaneous time-scale characteristic measures of the signal components causing bands C and D. This technique, which will be referred to herein as secondary wavelet feature decoupling (SWFD), may allow information concerning the nature of the signal components associated with the underlying physical process causing the primary band B (FIG. 3(c)) to be extracted when band B itself is obscured in the presence of noise or other erroneous signal features.

In some instances, an inverse continuous wavelet transform may be desired, such as when modifications to a scalogram (or modifications to the coefficients of a transformed signal) have been made in order to, for example, remove artifacts. In one embodiment, there is an inverse continuous wavelet transform which allows the original signal to be recovered from its wavelet transform by integrating over all scales and locations, a and b:

$$x ( t ) = \frac{1}{{C}_{g}} {\int }_{- ∞}^{∞} {\int }_{0}^{∞} T ( a , b ) \frac{1}{\sqrt{a}} ψ ( \frac{t - b}{a} ) \frac{d a d b}{{a}^{2}} \qquad ( 15 )$$

which may also be written as:

$$x ( t ) = \frac{1}{{C}_{g}} {\int }_{- ∞}^{∞} {\int }_{0}^{∞} T ( a , b ) {ψ}_{a , b} ( t ) \frac{d a d b}{{a}^{2}} \qquad ( 16 )$$

where C_{g} is a scalar value known as the admissibility constant. It is wavelet type dependent and may be calculated from:

$${C}_{g} = {\int }_{0}^{∞} \frac{{\vert \hat{ψ} ( f ) \vert}^{2}}{f} d f \qquad ( 17 )$$

FIG. 3(e) is a flow chart of illustrative steps that may be taken to perform an inverse continuous wavelet transform in accordance with the above discussion. An approximation to the inverse transform may be made by considering equation (15) to be a series of convolutions across scales. It shall be understood that there is no complex conjugate here, unlike for the cross correlations of the forward transform. As well as integrating over all of a and b for each time t, this equation may also take advantage of the convolution theorem which allows the inverse wavelet transform to be executed using a series of multiplications. FIG. 3(f) is a flow chart of illustrative steps that may be taken to perform an approximation of an inverse continuous wavelet transform. It will be understood that any other suitable technique for performing an inverse continuous wavelet transform may be used in accordance with the present disclosure.

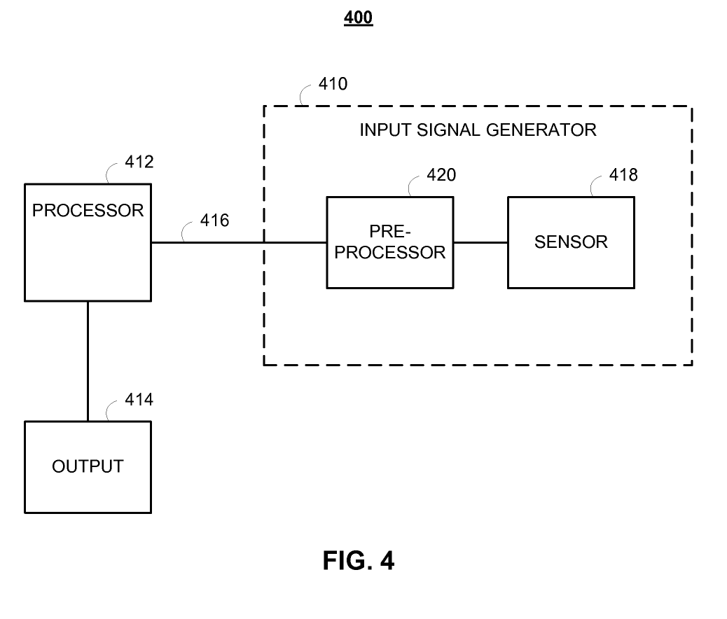

FIG. 4 is an illustrative continuous wavelet processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the continuous wavelet transforms of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof. Processor 416 may perform any suitable computation for signal analysis. For example, processor 416 may be capable of tuning a wavelet transform. In an embodiment, processor 416 tunes the wavelet transform to a particular characteristic frequency to produce better definition in the wavelet transform for repeating signal features.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to a continuous wavelet transform of input signal 416, such as data representing a scalogram. In one embodiment, data representing a scalogram may be stored in RAM or memory internal to processor 412 as any suitable three-dimensional data structure such as a three-dimensional array that represents the scalogram as energy levels in a time-scale plane. Any other suitable data structure may be used to store data representing a scalogram.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

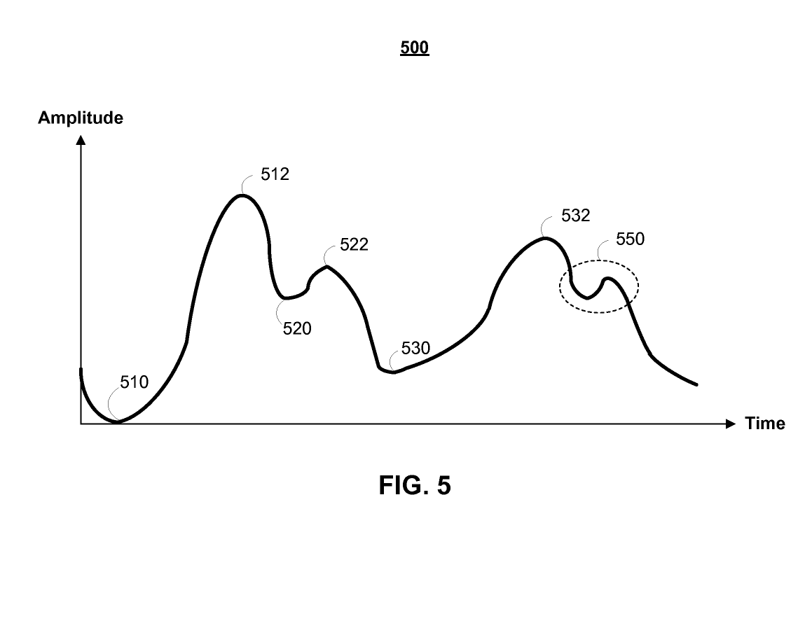

An illustrative PPG signal 500 is depicted in FIG. 5. Processor 412 may receive PPG signal 500, and may identify local minimum point 510, local maximum point 512, local minimum point 520, and local maximum point 522 in the PPG signal 500. Processor 412 may pair each local minimum point with an adjacent maximum point. For example, processor 412 may pair points 510 and 512 to identify one segment, points 512 and 520 to identify a second segment, points 520 and 522 to identify a third segment and points 522 and 530 to identify a fourth segment. The slope of each segment may be measured to determine whether the segment corresponds to an upstroke portion of the pulse (e.g., a positive slope) or a downstroke portion of the pulse (e.g., a negative slope) portion of the pulse. A pulse may be defined as a combination of at least one upstroke and one downstroke. For example, the segment identified by points 510 and 512 and the segment identified by points 512 and 520 may define a pulse.

According to an embodiment, PPG signal 500 may include a dichrotic notch 550 or other notches (not shown) in different sections of the pulse (e.g., at the beginning (referred to as an ankle notch), in the middle (referred to as a dichrotic notch), or near the top (referred to as a shoulder notch)). Processor 412 may identify notches and either utilize or ignore them when detecting the pulse locations. In some embodiments, processor 412 may compute the second derivative of the PPG signal to find the local minima and maxima points and may use this information to determine a location of, for example, a dichrotic notch. Additionally, processor 412 may interpolate between points in signal 416 or between points in a processed signal using any interpolation technique (e.g., zero-order hold, linear interpolation, and/or higher-order interpolation techniques). Some pulse detection techniques that may be performed by processor 412 are described in more detail in co-pending, commonly assigned U.S. patent application Ser. No. 12/242,908, filed Sep. 30, 2008 and entitled “SYSTEMS AND METHODS FOR DETECTING PULSES IN A PPG SIGNAL,” which is incorporated by reference herein in its entirety.

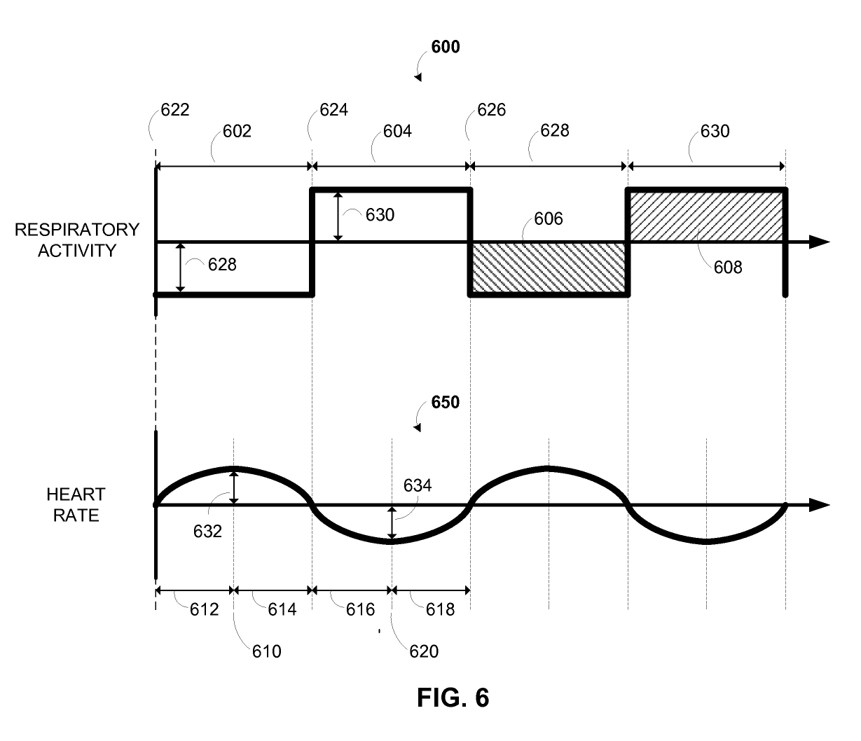

FIG. 6 shows exemplary plots that illustrate a relationship between respiratory activity and heart rate in accordance with an embodiment. Information about a patient's ANS may be determined based at least in part on this relationship. Plot 600 of respiratory activity and plot 650 of heart rate are illustrated over several cycles. The abscissa of the plots is a time axis common to both plots. It will be understood that the plots are merely illustrative and that actual patient-based plots may have different morphologies. The concepts being illustrated with respect to FIG. 6 will, however, apply to patient-based plots as well.

Plot 600 illustrates several respiration cycles which may correspond to either a respiration protocol, actual respiration of a patient, or a combination thereof. To the extent respiratory activity 600 is based on the actual respiration of a patient, it may be derived from any one or more suitable characteristics indicative of the patient's respiratory activity using any one or more suitable metrics. Such metrics include for example, length of inhalation, length of exhalation, volume of inhaled air, volume of exhaled air, airflow of inhalation, airflow of exhalation, any other suitable metric, or any combination thereof. In an embodiment, an ordinate of plot 600 of respiratory activity may indicate airflow, any other metric related to respiration, or any combination thereof. In an embodiment, area 606 indicates volume of inhaled air, and area 608 indicates volume of exhaled air. The four time segments (i.e., 602, 604, 628, 630) illustrated in plot 600 may correspond to two respiratory cycles. Each cycle may include inspiration and expiration. In an embodiment, time interval 602 corresponds to respiratory activity metrics below the abscissa and indicates length of inspiration, and time interval 604 corresponds to respiratory activity above the abscissa and indicates length of expiration. In an embodiment, amplitude 628 corresponds to local minimum airflow during inspiration and amplitude 630 corresponds to local maximum airflow during expiration.

Heart rate plot 650 is indicative of the heart rate of a patient and may be generated based at least in part on, for example, a PPG signal, electrocardiogram (EKG) signal, cardiophonogram signal, any other suitable physiological signal or any combination thereof, using any suitable processing technique. For example, the physiological signal may be generated at least in part by performing a wavelet transform or frequency domain transform of a PPG signal. In some embodiments, RSA (i.e., modulation of heart rate by respiratory activity) may be observed as a relationship between variation of heart rate in plot 650 in response to variation of respiratory activity in plot 600. For example, the increase in heart rate at time 610 occurs after an onset of inspiration at time 622, and the decrease of heart rate at time 620 occurs after an onset of expiration at time 624.

FIG. 6 illustrates some metrics which may provide information about a patient's ANS. The metrics include, for example, amplitude of a heart rate modulated by respiratory activity, a time delay between a respiratory event and a resultant effect on heart rate, or any combination thereof. An amplitude of heart rate modulation is indicated by amplitude 632 corresponding to inspiration, and amplitude 634 corresponding to expiration. Amplitude metrics may vary in response to respiration activity. For example, abrupt change in respiration rate may change the amplitude of heart rate modulation.

The time delays may be derived from phase relationships between fiducial points in the plot 600 of respiratory activity and plot 650 of heart rate. Phase relationships may be determined by examining derivatives, cross-correlations, auto-correlations, other calculated results from processed respiratory activity or processed heart rate information or any combination thereof. Fiducial points used for determining phase relationships may be identified by analyzing first or higher order derivatives of the curves in plots 600 and 650. For example in an embodiment, onset of heart rate increase or decrease may be identified from a first derivative of the curve in plot 650. Local minimum and maximum values at times 610 and 620 in heart rate plot 650 correspond to times when the first derivative of plot 650 is zero. In an embodiment, respiratory cycles may be determined from changes in concavity identified from a second derivative of the curve of respiratory activity in plot 600. Time intervals (i.e., 612, 614, 616 and 618) indicate examples of the time delay metric derived from phase relationships between fiducial points. Interval 612 indicates a phase difference between onset of inspiration at time 622 and change in heart rate at 610, and interval 616 indicates a phase difference between onset of expiration at time 624 and change in heart rate at time 620. Interval 614 indicates a phase difference between change in heart rate at time 610 and end of inspiration or onset of expiration at time 624. Interval 618 indicates a phase difference between change in heart rate and end of expiration or onset of inspiration at time 626. It should be understood that the metrics are merely illustrative and not limited to the described embodiments.

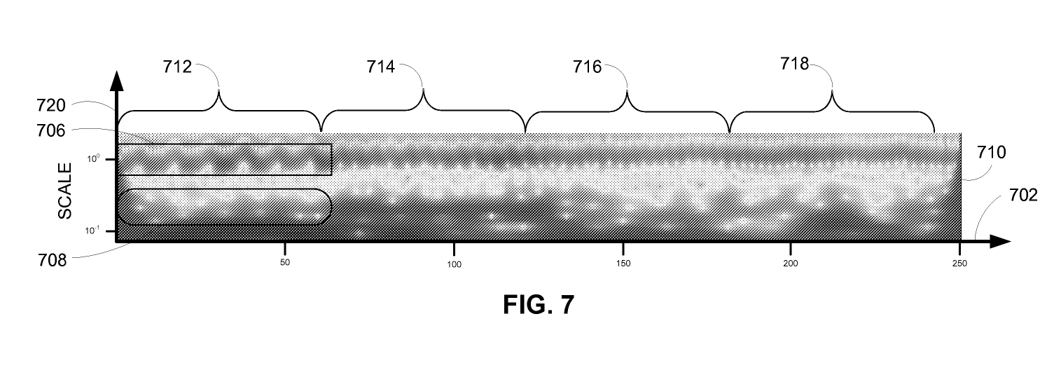

FIG. 7 is an illustrative scalogram showing a relationship between respiratory activity and heart rate in accordance with an embodiment. Abcissa 702 represents a time axis. Ordinate 720 represents scale. Scalogram 710 is generated based on a transformed physiological signal, such as a wavelet transformed PPG signal. Two illustrative regions of interest, indicated by band selection 706 and band selection 708, are selections of the pulse band and breathing band, respectively, corresponding to time interval 712. RSA is observed as the modulation of a ridge scale perturbation signal (e.g., RSP signal as described in reference to FIG. 3(d)) of the pulse band. For example, there are 6 oscillations in 1 minute time interval 712, corresponding to 6 breaths. As breathing rate increases from time interval 712 to 718, there is a reduction of the amplitude of the ridge scale signal perturbation and/or the ridge amplitude perturbation signal (e.g. RSP and RAP signals as described in reference to FIG. 3(d)). Respiratory activity information in plot 600 may be derived from breathing band 708, and heart rate information in plot 650 may be derived from pulse band 706. For example, heart rate information may be determined from an RSP signal based at least on the pulse band selection of the scalogram. In an embodiment, the respiratory activity may be derived from a respiration protocol, derived from the breathing band selection of the scalogram, or a physiological signal indicative of patient's respiration. For example, a curve indicating time of breaths may be determined from an RSP or RAP signal based at least in part on the breathing band selection 708 of the scalogram.

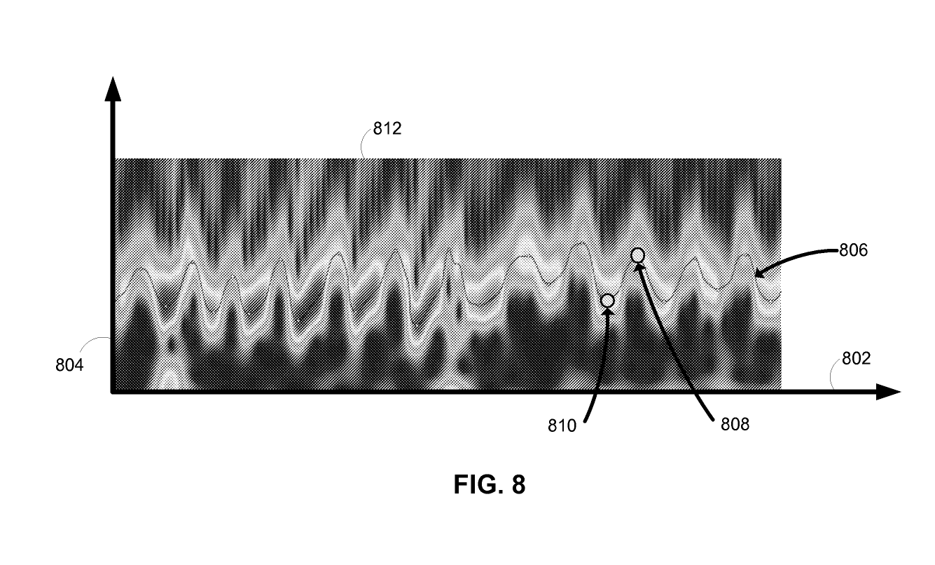

FIG. 8 is an illustrative scalogram showing a relationship between respiratory activity and heart rate in accordance with an embodiment. Scalogram 812 may be an enlarged view of a band selection from a scalogram generated from a wavelet transformed PPG signal. Several features are illustrated in the enlarged view. Abscissa 802 indicates a time axis, and ordinate 804 indicates a scale axis. Curve 806 indicates a ridge scale perturbation signal corresponding to heart rate and exhibits oscillations corresponding to RSA. Point 810 indicates a local minimum in an oscillation of the ridge scale signal, and point 808 indicates a local maximum in an oscillation of the ridge scale signal. Heart rate information may be determined from the scale of graph 806 to generate a plot of heart rate (e.g., plot 650 of FIG. 6) which may be used to extract metrics indicative of RSA as discussed above, for example.

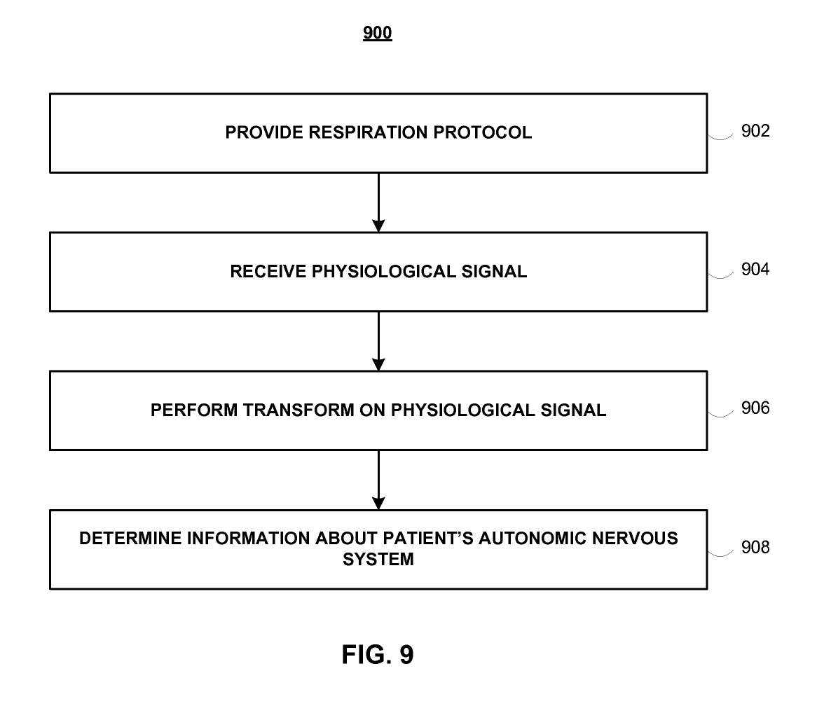

Flow chart 900 of FIG. 9 shows illustrative steps for determining information about a patient's ANS in accordance with an embodiment. The steps of flowchart 900 may be performed by any suitable processing device (e.g., a processor, digital processing device, analog hardware, or any combination thereof). It will be noted that the steps of flowchart 900 may be performed in any suitable order, and certain steps may be omitted, combined or otherwise altered.

In step 902, a respiration protocol may be implemented. A respiration protocol may be a suitable technique for controlling one or more aspects of a patient's respiratory activity. For example, a respiration protocol may include one or more instructions presented to a patient (e.g., by a monitor, by a clinician, by any other suitable device or person, or by any combination thereof) where the information provides an indication to the patient of how or when to breath. Instructions may include indications of when to inhale, exhale, or both; indications of how much air to inhale, exhale or both; indications of length of inhalation, exhalation, or both; indications of when to breath or when to hold one's breath; indications of respiration rate; indications of the amount of force to use in inhaling, exhaling, or both; indications of sitting or standing posture during breathing, indications of when to vary and how to vary any of these or other respiratory activities, any other indications to control any other suitable aspect of the patient's respiratory activity, or any combination thereof. In this disclosure, the terms inspiration and expiration may be used respectively in lieu of inhalation and exhalation, and vice versa. In some embodiments, a respiration protocol may be presented to a user in the form of visual cues, audio cues, tactile cues, any other suitable cues or a combination thereof. For example, a moving line on a two dimensional screen may indicate a respiration protocol. In a further example, an audible tone of rising pitch may correspond to inhalation, while an audible tone of falling pitch may correspond to exhalation. The length of the tones may correspond to the length of inhalation and length of exhalation. In some embodiments, a patient need not follow instructions of a respiration protocol (e.g., when a ventilator controls the patient's respiratory activity).

In step 904, at least one physiological signal of the patient may be received. The physiological signal may be received from any suitable source using any suitable technique. This signal may be a PPG signal, EKG signal, cardiophonographic signal, any other suitable physiological signal, or a combination thereof. The physiological signal may be responsive to physiological activity in the monitored patient such as heart rate, respiratory activity, or both. The received physiological signal may be generated by, for example, a sensor unit, which may itself include any of number of suitable physiological sensors, or a combination thereof. A received physiological signal may be pre-processed, post-processed, filtered, or any combination thereof and may include multiple physiological signals (e.g., first and second physiological signals), or multiple physiological signal components. For example, a received physiological signal may be a ratio of two physiological signals. In an embodiment, the physiological signals may include a Red PPG signal, an IR PPG signal, any other suitable physiological signal or any combination thereof. In an embodiment, a first and second physiological signal may be different types of physiological signals (e.g., indicative of blood pressure and pulse rate). In an embodiment, a first and second physiological signal may be obtained by first and second sensors located at approximately the same body site. In an embodiment, first and second physiological signals may be obtained by first and second sensors located at different body sites. It will be noted that the steps of flow chart 900 may be applied to any number of received physiological signals (e.g., PPG signals and physiological signals indicative of respiratory activity) by application of the techniques described herein.

In an embodiment, pre- or post-processing techniques may be applied to one or more of the physiological signals received at step 904. These techniques may include any one or more of the following: compressing, multiplexing, modulating, up-sampling, down-sampling, smoothing, calculating a statistic of the received physiological signal, removing erroneous regions of the received physiological signal, or any combination thereof.

In an embodiment, the at least one physiological signal received at step 904 may be filtered using any suitable filtering technique. For example, a physiological signal received from a sensor may be filtered by a low pass filter prior to undergoing additional processing at a microprocessor within a patient monitoring system. In some embodiments, a low pass filter may selectively remove frequencies that may later be ignored by a transformation or other processing step, which may advantageously reduce computational time and memory requirements. In an embodiment, a physiological signal received at step 904 may be high pass or band pass filtered (e.g., using a derivative filter) to remove low frequencies. In an embodiment, a physiological signal received at step 904 may be filtered to remove a DC component. In an embodiment, a physiological signal received at step 904 may be normalized by dividing the physiological signal by a DC component. In an embodiment, the cutoff frequencies of a filter may be chosen based on the frequency response of the hardware platform underlying the patient monitoring system.

Different operations, processing and/or filtering techniques, may be applied to any one or more of the physiological signals received at step 904 and/or any components of a multi-component signal. For example, different operations may be applied to a Red PPG signal and an IR PPG signal. An operation may be applied to a portion or portions of a received signal. An operation may be divided into one or more stages performed by one or more devices within a signal processing system (which may itself be a part of patient monitoring system).

In step 906, at least one physiological signal may be transformed to identify and extract metrics for determining information about the ANS. In an embodiment, a processor may transform the physiological signal into any suitable domain, for example, a Fourier, wavelet, spectral, scale, time, time-spectral, time-scale domain, any suitable transform space, or any combination thereof. This transformation may be performed by any one or more of the transformation techniques described herein, including a continuous wavelet transformation. For example, a PPG signal may be transformed using a continuous wavelet transform as described above with reference to FIG. 3(c). In an embodiment, a transformation may include performing a continuous wavelet transform for one or more PPG signals received, for example, at step 904, including an IR PPG signal, a Red PPG signal, or any combination of signals.

The continuous wavelet transform function may be based at least in part on a wavelet function. In an embodiment, the continuous wavelet transform utilizes a Morlet wavelet, a Mexican Hat wavelet, any suitable wavelet, or any combination thereof. This transformation may be performed by any suitable processing device, such as a processor, digital processing device, analog hardware or any combination thereof, which may be a general-purpose computing device, specialized processor, a separate dedicated device, or any combination thereof.

In step 908, information about a patient's ANS may be determined from at least one relationship between a transformed physiological signal and respiratory activity of the patient. Information about a patient's ANS may be determined from metrics of RSA parameterized by the respiration protocol provided in step 902. Metrics of RSA may be derived from amplitude and phase relationships between heart rate and respiratory activity. In an embodiment, the heart rate may be determined from a scalogram generated from a wavelet transformed PPG signal. For example, the heart rate may be based at least in part on the ridge scale perturbation signal of a pulse band selection from the scalogram.

Respiratory activity may be determined from the respiration protocol, a physiological signal indicative of the patient's respiration, or any combination thereof. In an embodiment, metrics of respiratory activity such as breathing rate, length of inhalation, length of exhalation, any other suitable metric of respiratory activity, or a combination thereof, may be determined from the respiration protocol described in step 902. In an embodiment, the metrics of respiratory activity may be determined from a physiological signal indicative of a patient's respiration. In an embodiment, metrics of respiratory activity may be determined from a breathing band of a scalogram generated from a wavelet transformed PPG signal. In an embodiment, a patient monitoring system and/or operator may provide a respiration protocol to a patient and may determine metrics of respiratory activity that are based at least in part on the respiration protocol and not based at least in part on a physiological signal indicative of respiration. In an embodiment, a patient monitoring system and/or operator may provide a respiration protocol to a patient and may determine metrics of respiratory activity that are based at least in part on the respiration protocol and a physiological signal indicative of respiration. In an embodiment, ventilator equipment may, according to a respiration protocol, control the breathing of the patient and may measure a physiological signal indicative of respiratory activity. For example, information about the times of breaths may be determined based at least on a ridge scale perturbation signal and/or ridge amplitude signal corresponding to a breathing band selection of the scalogram. In a further example, metrics of respiratory activity may be determined from the respiration protocol based at least in part on predetermined times, any other suitable characteristic of the respiration protocol, or any combination thereof. In a further example, a clinican, patient, any other suitable operator of the patient monitoring system, or any combination thereof, may indicate respiratory activity via a button push, any other suitable indications, or any combination thereof. The indications may be based at least in part on time of inhalation, time of exhalation, any other suitable metric of respiratory activity, or any combination thereof. In a further example, the metrics may be determined from a physiological signal indicative of respiration that may be received from a spirometer, thermistor, chest straps with transducers to measure movement, video image processing, ventilator output, airflow monitor coupled to a breathing mask or cannula, any other patient monitoring device capable of measuring respiratory activity, or a combination thereof.

Metrics of RSA indicative of information about a patient's ANS may be determined based on respiratory activity, and heart rate activity of the patient. In an embodiment, the respiratory activity may be determined based at least in part on a respiration protocol used in direct patient breathing, a measured physiological signal indicative of respiratory activity, or any combination thereof. In an embodiment, the heart rate activity may be determined based at least in part on a physiological signal affected by the patient. Illustrative metrics of RSA, such as time delay between onset of inspiration and change in heart rate, have been previously described in reference to FIG. 6. Analysis of scalograms generated from a transformed physiological signal has been described in reference to FIG. 7 and FIG. 8.

Metrics of RSA parameterized by at least one respiration protocol may be analyzed, compared to reference metrics, or both to determine information about the patient's ANS. In an embodiment, analysis of RSA metrics includes calculating a statistic of RSA metrics across several cycles, where the statistic may include maximum value, minimum value, mean, median, mode or any other suitable statistic or combination thereof. In some embodiments, parameterization of the respiration protocol may include gradual incrementing of breathing rate or breathing volume across time, abrupt changes in breathing rate or breathing volume across time, any other suitable variation of respiratory activity, or any combination thereof. The change in RSA metrics may be monitored to determine information about the patient's ANS. In an embodiment, the change in a phase difference between heart rate and respiratory activity or change in amplitude of heart rate may be monitored in response to steady increments in breathing rate across varying time intervals. For example, in FIG. 7, the breathing rate is gradually incremented from 6 breaths/minute to 24 breaths/minute across four 1 minute intervals. A reduction in the amplitude of ridge scale, corresponding to heart rate, is observed with increasing breathing rate. In an embodiment, change in a phase difference between heart rate and respiratory activity or change in amplitude of heart rate may be monitored in response to abrupt changes in breathing rate. It should be understood that the amplitude and phase difference metrics may be used alone or in combination in embodiments.

The metrics of RSA parameterized by respiration protocol may be compared to reference metrics related to the patient or reference metrics related to a group of patients. Parameterized metrics and reference metrics may be compared in a relative manner, absolute manner, or a combination thereof. Reference metrics may include metrics that indicate information about a patient's ANS, such as metrics of RSA which include phase differences of respiratory activity to heart rate, amplitude of heart rate modulation responsive to respiratory activity, any other suitable metric of RSA, or a combination thereof. Reference metrics may be stored on a memory of a measurement system, or accessed from a network storage system. In an embodiment, the reference metrics may be a history of metrics related to the patient, grouped by time periods, respiration protocol, mental and/or physical health conditions, any other suitable classifier, or any combination thereof. Comparison of parameterized metrics to patient related reference metrics may assess changes in the ANS of the patient compared to past history. In an embodiment, reference metrics may be related to a history of a group of patients. This group of patients may or may not include the patient, and may be selected based age, race, gender, weight, height, physical build, any other suitable characteristic related to the patient, or a combination thereof. The history may be grouped by time periods, respiration protocol, mental and physical health conditions, any other suitable classifier, or any combination thereof. The reference metrics related to the group of patients may be combined from the individual patient metrics by calculated a weighted average of metrics, or any other suitable aggregate metric.

In an embodiment, the parameterized metric may be compared to a reference group of metrics determined when the patient was in similar health condition, during similar time of day, other similar conditions, or a combination thereof to reduce sources of variability aside from the respiration protocol. In some embodiment, the comparison of parameterized metrics and reference metrics may diagnose the health condition of the patient. For example, an ailment may be identified by comparing the parameterized metric against reference metrics parameterized under similar respiration protocol and measured under similar conditions.

It will be understood that the systems and methods described herein include any combination of the above-described embodiments. Additionally, the systems and methods described herein (e.g., systems for implementing the steps illustrated in one or more of flow chart 600) may be applied to time domain signals, wavelet domain signals, signals in any suitable domain, or any combination thereof. It will also be understood that the above method may be implemented using any human-readable or machine-readable instructions on any suitable system or apparatus, such as those described herein.

The foregoing is merely illustrative of the principles of this disclosure and various modifications can be made by those skilled in the art without departing from the scope and spirit of the disclosure. The following claims may also describe various aspects of this disclosure.

What is claimed is:

1. A method for determining physiological information about a subject, the method comprising:
receiving a physiological signal affected by a patient's respiration, wherein at least one characteristic of the patient's respiration is known;
transforming the physiological signal using processing equipment into a transformed signal; and
determining using the processing equipment, information about the patient's autonomic nervous system based at least in part on the transformed signal and the at least one characteristic of the patient's respiration.

2. The method of claim 1, wherein the physiological signal is a photoplethysmograph signal.

3. The method of claim 1, wherein a known characteristic of the patient's respiration is derived from a respiration protocol.

4. The method of claim 1, wherein the at least one characteristic of the patient's respiration comprises respiration rate, volume of inhalation, volume of exhalation, length of inhalation, length of exhalation, time between inhalation and exhalation, time of inhalation, and/or time of exhalation, and/or a combination thereof.

5. The method of claim 1, wherein the transforming the physiological signal comprises transforming the physiological signal using a wavelet transform; and further comprising generating a scalogram based at least in part on the transformed signal.

6. The method of claim 1, wherein the determining is based at least in part on respiratory sinus arrhythmia.

7. The method of claim 1, wherein the determining comprises analyzing the transformed signal to identify the at least one metric associated with the patient's autonomic nervous system response.

8. The method of claim 7, wherein the at least one metric comprises a time delay between the patient's respiratory activity and a corresponding change in morphology of the transformed signal associated with the patient's autonomic nervous system response to the respiratory activity.

9. A system for determining physiological information about a subject, the system comprising:
an input configured to receive a physiological signal being affected by a patient's respiration, wherein at least one characteristic of the patient's respiration is known;
processing equipment configured to:
transform the physiological signal into a transformed signal; and
determine, based at least in part on the transformed signal and the at least one characteristic of the patient's respiration, information about the patient's autonomic nervous system.

10. The system of claim 9, wherein the physiological signal is a photoplethysmograph signal.

11. The system of claim 9, wherein a known characteristic of the patient's respiration is derived from a respiration protocol.

12. The system of claim 9, wherein the at least one characteristic of the patient's respiration comprises respiration rate, volume of inhalation, volume of exhalation, length of inhalation, length of exhalation, time between inhalation and exhalation, time of inhalation, and/or time of exhalation, and/or a combination thereof.

13. The system of claim 9, wherein the processing equipment is configured to transform the physiological signal using a wavelet transform; and to generate a scalogram based at least in part on the transformed signal.

14. The system of claim 9, wherein the processing equipment is configured to determine information about the patient's autonomic nervous system based at least in part on respiratory sinus arrhythmia.

15. The system of claim 13, wherein the processing equipment is configured to determine information about a patient's autonomic nervous system by analyzing the transformed signal to identify the at least one metric associated with the patient's autonomic nervous system response.

16. The system of claim 15, wherein the metric comprises a time delay between the patient's respiratory activity and a corresponding change in morphology of the transformed signal associated with the patient's autonomic nervous system response to the respiratory activity.

17. A computer-readable medium having stored instructions that when executed direct:
an input port to receive a physiological signal affected by a patient's respiration, wherein at least one characteristic of the patient's respiration is known; and
processing equipment to:
transform the physiological signal into a transformed signal; and
determine, based at least in part on the transformed signal and the at least one characteristic of the patient's respiration, information about the patient's autonomic nervous system.

18. The computer-readable medium of claim 17, wherein the physiological signal is a photoplethysmograph signal.

19. The computer-readable medium of claim 17, wherein a known characteristic of the patient's respiration is derived from a respiration protocol.

20. The computer-readable medium of claim 17, wherein the at least one characteristic of the patient's respiration comprises respiration rate, volume of inhalation, volume of exhalation, length of inhalation, length of exhalation, time between inhalation and exhalation, time of inhalation, and/or time of exhalation, and/or a combination thereof.

21. The computer-readable medium of claim 17, wherein the processing equipment is directed to transform the physiological signal using a wavelet transform; and to generate a scalogram based at least in part on the transformed signal.

22. The computer-readable medium of claim 17, wherein the processing equipment is directed to determine information about the patient's autonomic nervous system based at least in part on respiratory sinus arrhythmia.

23. The computer-readable medium of claim 21, wherein the determining comprises analyzing the transformed signal to identify at least one metric associated with the patient's autonomic nervous system response; and wherein the metric comprises a time delay between the patient's respiratory activity and a corresponding change in morphology of the transformed signal associated with the patient's autonomic nervous system response to the respiratory activity.
